(19) United States
(12) Patent Application Publication
(10) Pub. No.: US 2010/0310544 A1
(43) Pub. Date: Dec. 9, 2010

(54) COMPOSITION COMPRISING CALCIUM IONS AND AT LEAST ONE PROTEOLYTIC ENZYME FOR USE IN THE IN VITRO AND IN VIVO REGENERATION OF CUTANEOUS AND CONNECTIVE TISSUES

(75) Inventors: Luisa Gennero, Torino (IT); Chiara Cesano, Torino (IT); Carlo Vercelli, Torino (IT); Antonio Ponzetto, Torino (IT); Gianfranco Merizzi, Torino (IT)

Correspondence Address: Steinfl & Bruno, 301 N Lake Ave Ste 810, Pasadena, CA, 91101, US

(73) Assignee: MEDEVICE S.P.A., TORINO (IT)

(21) Appl. No.: 12/745,253

(22) PCT Filed: Oct. 27, 2008
(86) PCT No.: PCT/IB08/54423

§ 371 Date: May 27, 2010

(30) Foreign Application Priority Data
Nov. 30, 2007 (IT) TO2007A000870

(51) Int. Cl. A61K 38/51; A61K 38/48; A61P 17/02; A61P 43/00; A61Q 19/08; A61Q 19/00; C12N 5/07

(52) U.S. Cl. 424/94.5; 424/94.64; 424/94.63; 424/94.65; 424/94.67; 435/404

(57) ABSTRACT

The invention is based on the finding that calcium ions and proteolytic enzymes, particularly papain, act in a synergetic way in the regeneration of skin and connective tissues. Thus, such a combination of active principles can be used for the preparation of culture media for cutaneous and/or connective tissue biopsies, which allow for the maintenance of the biopsies under viable conditions for periods of over 6 months, even for over 3 years, as well as for the preparation of pharmaceutical compositions or medical devices for the treatment of diseases in which the regeneration of cutaneous and connective tissues is beneficial (for instance, injuries and wounds, keloids, paradontopathies, conjunctivitis, nasal and ear polyps, cellulite, human and animal dermatitis), and for the preparation of cosmetic compositions that in particular have an anti-ageing effect on the skin.

The present invention relates to a composition comprising the combination of at least two active principles, the said composition being particularly effective in the regeneration and repairing of the cutaneous, mucous and connective tissues and amenable to be prepared in various physical forms and formulations specifically adapted to the specific intended use. In particular, the composition of the invention can be prepared and used as a cell culture medium, cosmetic composition, pharmaceutical composition, medical device, for human or veterinary use.

As will be described in detail in the following experimental section, the composition of the invention proved to be effective in the regeneration and repairing of cutaneous, mucous and connective tissues both in vitro and in vivo, by creating the optimum physiological and biochemical conditions in order to stimulate the viability and trophism of tissues, especially damaged tissues, and in the regeneration of native stem cells and the physiological differentiation thereof.

The experiments performed in vitro demonstrate that the composition of the invention has a considerable regenerating activity for cells and tissues, such as to advantageously allow for the maintenance of cultured tissue biopsies under viable conditions for at least 6 months, even for over 3 years.

In this respect, the work by Robinson M et al. [1] is mentioned, which concerns in vitro cultures of tissues containing follicles from adult rat whiskers. This study has set the basis for the establishment of a culture protocol that is able to keep the hair follicles viable for not over 1 month. The histological examination of the samples at 20 and 30 days showed that tissue necrosis occurred after about one month of continuous culture in vitro. Prior to this research, the in vitro survival of such tissues generally ceased after a few days of culture (5-10 days).

In the light of the in vitro regenerative abilities, the composition of the invention is therefore particularly suitable as a culture medium designed to maintain tissue biopsies under viable conditions for an extremely sustained period.

Since the regenerative and repairing proprieties detected in vitro are maintained in vivo too, the composition of the invention further lends itself to diverse in vivo applications, including cosmetic applications, particularly as an anti-ageing, anti-blemish, moisturising, nourishing, elasticising, soothing, regenerating, anti-cellulite and anti-stretch mark cosmetic product, as well as a pharmaceutical product with cicatrizant and/or regenerating activity, particularly for the treatment of human or animal medical pathologies or conditions, such as injuries, wounds, keloids, paradontopathies, nasal polyps, pyotraumatic dermatitis, scrotal ulcerative dermatitis, acral lick dermatitis, self-traumatic dermatitis of head and neck.

In the scope of the present description, the term “connective tissues” particularly means subcutaneous tissue, mucous tissue, loose connective tissue, dense connective tissue, reticular tissue, elastic tissue, lymphoid tissue, and fat tissue. The term “cutaneous tissues” is understood to mean the integument system in its entirety, which comprises both the skin, including the scalp, and the skin annexes, such as for example the hair.

### SUMMARY OF THE INVENTION

One object of the present invention is to provide a composition capable of inducing the regeneration and repairing of human and animal cutaneous, mucous and connective tissues in a particularly effective way, both in vitro and in vivo, and therefore suitable for diverse types of applications, among which cosmetic and pharmaceutical applications, and as a culture medium suitable to maintain viable conditions in tissue biopsies for very long periods.

These and other objects are attained by the composition as defined in the attached claims. The claims are an integral part of the description.

The composition of the invention is based on the combination of calcium ions and one proteolytic enzyme, preferably papain.

Depending on the intended applications and uses, the composition of the invention can be provided in the form of a pharmaceutical composition (in many tissue-specific formulations), a medical device, a cosmetic composition or a cell and/or tissue culture medium.

As will be described in greater detail in the experimental section, the composition of the invention based on the combination of calcium ions and a proteolytic enzyme was tested both in vitro and in vivo. The results obtained confirm the regeneration and re-trophisation of cutaneous, subcutaneous, mucous and connective tissues in general. The histological results obtained in vitro after 6 months of treatment are particularly significant. The tissue re-trophisation induced by the composition of the invention is morphologically comparable to the trophic condition of intact tissues in vivo, with optimum histo-functional characteristics.

### DETAILED DESCRIPTION OF THE INVENTION

The present invention provides a composition capable of inducing a regenerating activity in cells and tissues in vitro, such as to allow for the maintenance of cultured tissue biopsies under viable conditions for at least 6 months, and to quicken tissue repairing. Moreover, the composition is suitable for in vivo applications, both in the cosmetic and the pharmaceutical fields.

The composition of the invention is capable of stimulating the growth of epidermal, dermal, mucous and connective tissues in general, improving the viability and trophism of these tissues, especially if damaged, through establishment of conditions suitable for favouring the regeneration and differentiation of native and non-native pluripotent stem cells.

The invention is based on the finding of a particular pro-proliferative stimulus carried out by the combination of calcium ions and proteolytic agents, such as for example papain, on cutaneous, mucous, subcutis and connective tissues in general.

Calcium ions activate the coagulation process (thrombin becomes activated in the presence of calcium ions by the reaction of phospholipids released from the cells of damaged tissues and from the clustered platelets), with an afflux of platelets into the dermis and the subsequent spontaneous release of various growth factors therefrom. Parallel to the action induced by calcium ions, the proteolytic enzyme (that preferably is papain) promotes two essential functions, that is: (i) the activation of the growth factors physiologically present in the micro-environment of damaged or senescent tissues, and (ii) the activation of the same platelet growth factors attracted by the calcium ions, with a consequent chemotactic induction of the resident stem compartment towards a rapid repairing differentiation. Chemotaxis is the process whereby a cell is able to detect chemical extracellular signal gradients and move in the direction of the concentration gradient. Chemotaxis plays an essential role in development, in the immune system and in tissue regeneration.

Furthermore, calcium ions and papain together carry out two synergetic functions, that is: (i) calcium ions in the form of salts bring about a micro-desiccation and a surface exfoliation of the wounds, eliminating the seriously damaged cells and correcting the peripheral dysemia (unbalance of mineral salts in the blood that flows into the dermal capillaries after a lesion) that occurs in the dermis after a trauma; (ii) papain accelerates the absorption of the nutrients present in the preparation. Moreover, high doses of papain stimulate the Langerhans' cells (spinous layer) to incorporate and digest the foreign substances (excess of collagen). Such a mechanism turned out to be particularly useful in the treatment of keloids. It is also known that papain, besides performing a keratolytic function, has also exfoliation and antimicrobial properties in the epidermis, facilitating the elimination of necrotic tissue, the formation of granulation tissue and preventing viral or bacterial over-infection phenomena at the site of the lesion. Furthermore, papain assists in the treatment of both post-traumatic and inflammatory edemas, through a proteolytic mechanism on fibrin, favouring the drainage of the inflammatory focus and the resorption of the haemorrhagic extravasation.

Such findings have led to the generation of a composition suitable both for the use as a culture medium in vitro and for applications in vivo, based on the combination of calcium ions (Ca²⁺) (introduced into the composition in the form of calcium salts, preferably as calcium chloride) and at least one proteolytic enzyme, preferably papain, in an effective concentration.

Thus, the object of the invention is a composition comprising, in a physiologically acceptable carrier, the usual amino acids, sugars, salts and vitamins for the in vitro culture of eukaryotic cells or tissues, characterised in that it further comprises at least one proteolytic enzyme and a calcium salt, the calcium salt being present in the composition with a concentration of calcium ions of at least 3.6 mM.

The said composition displays a regenerating activity on cells and tissues such as to allow for the maintenance of cultured tissue biopsies under viable conditions for at least 6 months and to accelerate tissue repairing.

The histological results obtained in vitro after treatment with the composition of the invention confirm the induction of regeneration and re-trophisation of skin, skin annexes, subcutis, mucous and connective tissues, which appear morphologically comparable to in vivo intact tissues and with optimum histo-functional characteristics (see Example 1).

The in vitro treatment of senescent or damaged biopsy tissue with the composition of the invention results in an amazing repairing of the damages and a total recovery of the original trophism. All of the tissues in the treated biopsy samples undergo a complete functional and morphological regeneration, consequent to a possible absence or reduced presence of the ageing processes.

The histological results obtained in vivo starting from 7 days of treatment with the composition forming the subject of the present invention and its tissue-specific formulations (see the section related to the Clinical Studies) confirm, by histological examination, the formation of a regeneration and re-trophisation of skin, subcutis, mucous and connective tissues in general, with optimum histo-functional characteristics morphologically comparable to in vivo intact skin, subcutaneous, mucous and connective tissues.

In addition to the ingredients mentioned above, the composition of the invention can comprise further active ingredients, selected for their enhancing and synergetic activity on the two above-defined active principles, also as a function of the tissue type to be treated.

The following tables, which are provided for illustration purpose only and not as a limitation, represent embodiments of the composition of the invention, each specifically designed for a particular tissue and a particular application type.

The compositions provided in the tables can be modified without departing from the scope of the invention. For instance, a different calcium salt can be used instead of calcium chloride, such as for example calcium gluconate, calcium phosphate, calcium nitrate, provided that the concentration of calcium ions (Ca²⁺) is at least 3.6 mM. The preferred concentration of calcium ions is in the range from 7 mM to 27 mM.

Papain is preferred as the proteolytic enzyme. As an alternative to papain, one can use collagenase, Serratiopeptidase, heparanase, elastase, bromelain, bradykinase, Clostridium peptidase, enzymes expressed by Lactobacillus acidophilus, enzymes expressed by the genus Aspergillus, protease, aliinase, fibrinolysin. The amount of proteolytic enzyme can be varied and adapted to the specific relevant requirements, taking into account the specific proteolytic enzyme of choice and the desired proteolytic activity, without requiring the exercise of any inventive skills.

If the composition is a cosmetic, pharmaceutical or medical device composition designed for the treatment of the oral mucosa or the parodontal tissue, the preferred concentration of calcium ions is in the range from 500 to 1500 mg/L (or mg/kg in the case of a solid composition) and the preferred concentration of the proteolytic enzyme, preferably papain, is in the range from 0.1 to 20 mg/L (or mg/kg).

If the composition is a cosmetic, pharmaceutical or medical device composition designed for the treatment of the nasal mucosa, the preferred concentration of calcium ions is in the range from 500 to 1500 mg/L (or mg/kg in the case of a solid composition) and the preferred concentration of the proteolytic enzyme, preferably papain, is in the range from 0.1 to 10 mg/L (or mg/kg).

If the composition is a cosmetic, pharmaceutical or medical device composition designed for the treatment of skin and subcutis, the preferred concentration of calcium ions is in the range from 500 to 1500 mg/L (or mg/kg in the case of a solid composition) and the preferred concentration of the proteolytic enzyme, preferably papain, is in the range from 5 to 50 mg/L (or mg/kg).

If the composition is a culture medium, the preferred concentration of calcium ions is in the range from 500 to 1500 mg/L and the preferred concentration of the proteolytic enzyme, preferably papain, is in the range from 5 to 50 mg/L.

The nature and the concentrations of the additional ingredients can be varied too without departing from the scope of the invention. For instance, the retinoic acid concentration may vary within the range from about 0.001 to about 1 mg/L (or mg/kg); the dexamethasone concentration may vary within the range from about 10⁻⁴ to about 10⁻⁸ M; the scopolamine concentration may vary within the range from about 0.1 to about 50 mg/L (or mg/kg); the hyaluronic acid concentration may vary within the range from about 100 mg/L to about 15 g/L (or g/kg); the fatty acid concentration, among which butyric acid is preferred, may vary within the range from about 0.01 to about 2% w/v; the GABA concentration may vary within the range from about 0.01 to about 2% w/v; the carnithine concentration may vary within the range from about 0.1 to about 10% w/v; the lauric acid concentration may vary within the range from about 0.01 to about 2% w/v; the camphor oil concentration may vary within the range from about 0.01 to about 2% w/v. The above-mentioned ingredients are not compulsory in the composition of the present invention, therefore one or more than one of them may be absent or replaced totally or in part by other ingredients.

The compositions of the invention upon testing have given optimum results in terms of growth and development of cutaneous, subcutaneous, mucous and connective tissues, with normal histo-functional characteristics. The compositions of the invention, prepared as culture media, have been further tested to assess their effectiveness in the maintenance of the viability of biopsy samples. All of the cutaneous and connective tissue biopsy samples tested responded positively to the use of the culture media of the invention, by remaining viable, depositing matrix and displaying an ordered and three-dimensional distribution for at least six months of culture in vitro, compared to the 20-30 days of lifetime attainable with the normal culture media. Without wishing to be bound by any specific theory on the matter, the present inventors believe that the results obtained with the culture media that are the subject of the present invention have allowed to demonstrate that the atrophy status of the skin during degenerative processes is recoverable.

### Examples of Tissue-Specific Compositions

### 1) Oral Mucous and Parodontal Tissues

### QParodontal-BASE Composition

| Substance | Concentration | |
|---|---|---|
| Active principles | | |
| Calcium Chloride | 1200 | mg/L |
| Papain FU | 2.00 | mg/L |
| Excipients | | |
| Glycyrrhetic acid | 2000 | mg/L |
| Calcium Gluconate | 100 | mg/L |
| Distilled water | q.s. up to 1 | L of solution |

### 2) Oral Mucous and Parodontal Tissues

### QParodontal-BASE-Plus Composition

| Substance | Concentration | |
|---|---|---|
| Active principles | | |
| Calcium Chloride | 1200 | mg/L |
| Papain FU | 2.00 | mg/L |
| Excipients | | |
| Glycyrrhetic acid | 2000 | mg/L |
| Calcium Gluconate | 100 | mg/L |
| Propolis in hydro-alcoholic sol. (15 ml) | 45 ml for 1 L of solution | |
| Distilled water | q.s. up to 1 | L of solution |

### 3) Oral Mucous and Parodontal Tissues

### QParodontal-BASE-Gel Composition

| Substance | Concentration | |
|---|---|---|
| Active principles | | |
| Calcium Chloride | 1200 | mg/L |
| Papain FU | 2.00 | mg/L |
| Excipients | | |
| Glycyrrhetic acid | 2000 | mg/L |
| Calcium Gluconate | 100 | mg/L |
| Cellulose powder | 60 | g/L (6% solution) |
| Distilled water | q.s. up to 1 | L of solution |

### 4) Oral Mucous and Parodontal Tissues

### Q-Parodontal-BASE-GelPlus Composition

| Substance | Concentration |
|---|---|
| Active principles | |
| Calcium Chloride | 1200 mg/L |
| Papain FU | 2.00 mg/L |
| Excipients | |
| Glycyrrhetic acid | 2000 mg/L |
| Calcium Gluconate | 100 mg/L |
| Cellulose powder | 60 g/L (6% solution) |
| Propolis in hydro-alcoholic sol. (15 ml) | 45 ml for 1 L of solution |
| Distilled water | q.s. up to 1 L of solution |

### 5) Oral Mucous and Parodontal Tissues

### QParodontal-Hyal Composition

| Substance | Concentration | |
|---|---|---|
| Active principles | | |
| Calcium Chloride | 1200 | mg/L |
| Papain FU | 2.00 | mg/L |
| Hyaluronic acid | 100.00 | mg/L (0.1 g/L) |
| Glycyrrhetic acid | 2000 | mg/L |
| Calcium Gluconate | 100 | mg/L |
| Distilled water | q.s. up to 1 | L of solution |

### 6) Oral Mucous and Parodontal Tissues

### QParodontal-Hyal-Plus Composition

| Substance | Concentration | |
|---|---|---|
| Active principles | | |
| Calcium Chloride | 1200 | mg/L |
| Papain FU | 2.00 | mg/L |
| Hyaluronic acid | 100.00 | mg/L (0.1 g/L) |
| Glycyrrhetic acid | 2000 | mg/L |
| Calcium Gluconate | 100 | mg/L |
| Propolis in hydro-alcoholic sol. (15 ml) | 45 ml for 1 L of solution | |
| Distilled water | q.s. up to 1 | L of solution |

### 7) Oral Mucous and Parodontal Tissues

### QParodontal-Hyal-Gel Composition

| Substance | |
|---|---|
| Active principles | Concentration |
| Calcium Chloride | 1200 mg/L |
| Papain FU | 2.00 mg/L |
| Hyaluronic acid | 10000.00 mg/L (10 g/L) |
| Glycyrrhetic acid | 2000 mg/L |
| Calcium Gluconate | 100 mg/L |
| Cellulose powder | 60 g/L (6% solution) |
| Distilled water | q.s. up to 1 L of solution |

### 8) Oral Mucous and Parodontal Tissues

### Q-Parodontal-Hyal-GelPlus Composition

| Substance | Concentration | |
|---|---|---|
| Active principles | | |
| Calcium Chloride | 1200 | mg/L |
| Papain FU | 2.00 | mg/L |
| Hyaluronic acid | 10000.00 | mg/L (10 g/L) |
| Glycyrrhetic acid | 2000 | mg/L |
| Calcium Gluconate | 100 | mg/L |
| Cellulose powder | 60 | g/L (6% solution) |
| Propolis in hydro-alcoholic sol. (15 ml) | 45 ml for 1 L of solution | |
| Distilled water | q.s. up to 1 | L of solution |

### 9) Nasal Mucous Tissue

### QNasal-BASE Composition

| Substance | Concentration | |
|---|---|---|
| Active principles | | |
| Calcium Chloride | 1200 | mg/kg |
| Papain FU | 0.20 | mg/kg |
| Excipients | | |
| Sodium phosphate, dibasic | 0.18 | g/kg |
| Sodium phosphate, monobasic | 0.17 | g/kg |
| Distilled water | q.s. up to 1 | L of solution |

### 10) Cutaneous and Subcutaneous Tissues

### Q-BASE-MD GEL Composition (Medical Device)

| Substance | Concentration | |
|---|---|---|
| Calcium Chloride | 1100 | mg/kg |
| Papain FU | 22.00 | mg/kg |
| Dexamethasone 21-phosphate disodium | 44 μg/kg or 10⁻⁷M | |
| Scopolamine (Butyl bromide) | 4 | mg/Kg |
| Retinoic Acid | 0.0020 | mg/kg |
| Hyaluronic Acid | 10,000 | mg/kg |
| Ascorbic Acid | 400.00 (2.2 gr/Kg) | |
| Silver proteinate | 400 | mg/kg |
| Natural L-amino acid mix | 28.5 | g/kg |

| | |
|---|---|
| BASE as a gel (carbopol or cellulose derivatives) | q.s. per kg of product |

### 11) Cutaneous and Subcutaneous Tissues

### Q-BASE-CREAM Composition

| Substance | Concentration | |
|---|---|---|
| Calcium Chloride | 1100 | mg/kg |
| Papain FU | 22.00 | mg/kg |
| Dexamethasone 21-phosphate disodium | 44 μg/kg or 10⁻⁷M | |
| Scopolamine (Butyl bromide) | 4 | mg/kg |
| Retinoic Acid | 0.0020 | mg/kg |
| Hyaluronic Acid | 10,000 | mg/kg |
| Ascorbic Acid | 400.00 | mg/kg |
| Silver proteinate | 400 | mg/kg |
| Natural L-amino acid mix | 28.5 | g/kg |

| | |
|---|---|
| BASE as a cream or emulsion (O/W or W/O) (such | q.s. per kg of |
| as: water, white vaseline, cetostearyl alcohol, liquid | product |
| paraffin, ceteth-20, sodium phosphate, p-chloro-m- | |
| cresol, phosphoric acid) | |

### 12) Cutaneous and Subcutaneous Tissues

### Q-BASE-INFUS Composition (Infusion Liquid)

| Substance | Concentration | |
|---|---|---|
| Calcium Chloride | 1100 | mg/L |
| Papain FU | 22.00 | mg/L |
| Dexamethasone 21-phosphate disodium | 44 μg/L or 10⁻⁷M | |
| Scopolamine (Butyl bromide) | 4 | mg/L |
| Retinoic Acid | 0.0020 | mg/L |
| Hyaluronic Acid | 100.00 | mg/L |
| Ascorbic Acid | 400.00 | mg/L |
| Silver proteinate | 400 (0.4 gr/L) | |
| Natural L-amino acid mix | 28.5 | g/L |

| | |
|---|---|
| Physiological solution | q.s. per L of solution |

### 13) Cutaneous and Subcutaneous Tissues

### QCell-BASE-CREAM Composition

| Substance | Concentration | |
|---|---|---|
| Calcium Chloride | 1100 | mg/kg |
| Papain FU | 22.00 | mg/kg |
| Dexamethasone 21-phosphate disodium | 44 μg/kg or 10⁻⁷M | |
| Scopolamine (Butyl bromide) | 4 | mg/kg |
| Gamma-Amino Butyric Acid | 0.06% | w/v total |
| Butyric Acid | 0.06% | w/v total |
| Retinoic Acid | 0.0020 | mg/kg |
| Hyaluronic Acid | 1000 | mg/kg |
| Ascorbic Acid | 400.00 (2.2 g/kg) | |
| Carnithine | 0.05% | w/v total |
| Natural L-amino acid mix | 28.5 | g/kg |

| | |
|---|---|
| BASE as a cream or emulsion (O/W or W/O) (such | q.s. per kg of |
| as: water, white vaseline, cetostearyl alcohol, liquid | product |
| paraffin, ceteth-20, sodium phosphate, p-chloro-m- | |
| cresol, phosphoric acid) | |

### 14) Cutaneous and Subcutaneous Tissues

### QCell-BASE-Gel Composition

| Substance | Concentration | |
|---|---|---|
| Calcium Chloride | 1100 | mg/kg |
| Papain FU | 22.00 | mg/kg |
| Dexamethasone 21-phosphate disodium | 44 μg/kg or 10⁻⁷M | |
| Scopolamine (Butyl bromide) | 4 | mg/kg |
| Gamma-Amino Butyric Acid | 0.06% | w/v total |
| Butyric Acid | 0.06% | w/v total |
| Retinoic Acid | 0.0020 | mg/kg |
| Hyaluronic Acid | 1000 | mg/kg |
| Ascorbic Acid | 400.00 | mg/kg |
| Carnithine | 0.05% | w/v total |
| Natural L-amino acid mix | 28.5 | g/kg |

| | |
|---|---|
| BASE as a gel (carbopol or cellulose derivatives) | q.s. per kg of product |

### 15) Cutaneous and Subcutaneous Tissues

### QCell-BASE-Infus Composition

| Substance | Concentration | |
|---|---|---|
| Calcium Chloride | 1100 | mg/L |
| Papain FU | 22.00 | mg/L |
| Dexamethasone 21-phosphate disodium | 44 μg/L or 10⁻⁷M | |
| Scopolamine (Butyl bromide) | 4 | mg/L |
| Gamma-Amino Butyric Acid | 0.06% | w/v total |
| Butyric Acid | 0.06% | w/v total |
| Retinoic Acid | 0.0020 | mg/L |
| Hyaluronic Acid | 100.00 | mg/L |
| Ascorbic Acid | 400.00 | mg/L |
| Carnithine | 0.05% | w/v total |
| Natural L-amino acid mix | 28.5 | g/L |

| | |
|---|---|
| Physiological solution | q.s. per L of product |

### 16) Cutaneous and/or Connective Tissue Biopsies

### Q-BASE Culture Medium

| Substance | Concentration | |
|---|---|---|
| Calcium Chloride | 1100 | mg/L |
| Papain FU | 22.00 | mg/L |
| Dexamethasone 21-phosphate disodium | 44 μg/L or 10⁻⁷M | |
| Scopolamine (Butyl bromide) | 4 | mg/L |
| Retinoic Acid | 0.0020 | mg/L |
| Hyaluronic Acid | 100.00 | mg/L |
| Ascorbic Acid | 400.00 | mg/L |
| Natural L-amino acid mix | 28.5 | g/L |
| F12 culture medium | 10 | ml |
| MEM non-essential amino acid solution | 20 | ml |
| HANK's solution | 2 | ml |
| Autologous serum or a substitute thereof | 4 | ml |
| D-MEM | q.s. to 1 | L of solution |

### 17) Cutaneous and/or Connective Tissue Biopsies

### Q-BASE-Gel Culture Medium

| | |
|---|---|
| Substance | Concentration |

| Calcium Chloride | 1100 | mg/L |
|---|---|---|
| Papain FU | 22.00 | mg/L |
| Dexamethasone 21-phosphate disodium | 44 μg/L or 10⁻⁷M | |
| Scopolamine (Butyl bromide) | 4 | mg/L |
| Retinoic Acid | 0.0020 | mg/L |
| Hyaluronic Acid | 100.00 | mg/L |
| Ascorbic Acid | 400.00 | mg/L |
| PDRN | 100 | mg/L |
| Natural L-amino acid mix | 28.5 | g/L |
| F12 culture medium | 10 | ml |
| MEM non-essential amino acid solution | 20 | ml |
| HANK'S solution | 2 | ml |
| Autologous serum or a substitute thereof | 4 | ml |

| | |
|---|---|
| D-MEM | q.s. to 1 L of solution |
| Collagen scaffold for gel cultures | q.s. |

### 18) Cutaneous and Subcutaneous Tissues

### QAntiAgeing-BASE-Gel Composition

| Substance | Concentration | |
|---|---|---|
| Calcium Chloride | 1100 | mg/Kg |
| Papain FU | 22.00 | mg/Kg |
| Dexamethasone 21-phosphate disodium | 44 μg/Kg or 10⁻⁷M | |
| Scopolamine (Butyl bromide) | 4 | mg/Kg |
| Gamma-Amino Butyric Acid | 0.06% | w/v total |
| Butyric Acid | 0.06% | w/v total |
| Retinoic Acid | 0.0020 | mg/kg |
| Hyaluronic Acid | 1000 | mg/kg |
| Ascorbic Acid | 400.00 | mg/kg |
| Carnithine | 0.05% | w/v total |
| PDRN | 0.1% | w/v total |

| | |
|---|---|
| BASE as a gel (carbopol or cellulose derivatives) | q.s. per kg of product |

### 19) Cutaneous and Subcutaneous Tissues

### QAntiAgeing-BASE-CREAM Composition

| Substance | Concentration | |
|---|---|---|
| Calcium Chloride | 1100 | mg/kg |
| Papain FU | 22.00 | mg/kg |
| Dexamethasone 21-phosphate disodium | 44 μg/kg or 10⁻⁷M | |
| Scopolamine (Butyl bromide) | 4 | mg/kg |
| Gamma-Amino Butyric Acid | 0.06% w/v total | |
| Butyric Acid | 0.06% w/v total | |
| Retinoic Acid | 0.0020 | mg/kg |
| Hyaluronic Acid | 1000 | mg/kg |
| Ascorbic Acid | 400.00 | mg/kg |

| | |
|---|---|
| Carnithine | 0.05% w/v total |
| PDRN | 0.1% w/v total |
| BASE as a cream or emulsion (O/W or W/O) | q.s. per kg of product |
| (such as: water, white vaseline, | |
| cetostearyl alcohol, liquid paraffin, | |
| ceteth-20, sodium phosphate, p-chloro- | |
| m-cresol, phosphoric acid) | |

### 20) Cutaneous and Subcutaneous Tissues

### QAntiAgeing-BASE-INFUS Composition

| Substance | Concentration | |
|---|---|---|
| Calcium Chloride | 1100 | mg/L |
| Papain FU | 22.00 | mg/L |
| Dexamethasone 21-phosphate disodium | 44 μg/L or 10⁻⁷M | |
| Scopolamine (Butyl bromide) | 4 | mg/L |
| Gamma-Amino Butyric Acid | 0.06% | w/v total |
| Butyric Acid | 0.06% | w/v total |
| Retinoic Acid | 0.0020 | mg/L |
| Hyaluronic Acid | 100.00 | mg/L |
| Ascorbic Acid | 400.00 | mg/L |
| Carnithine | 0.05% | w/v total |
| PDRN | 0.1% | w/v total |

| | |
|---|---|
| Physiological solution | q.s. per L of product |

### 21) Cutaneous and Subcutaneous Tissues

### QAntiAgeing-BASE-Lotion Composition

| Substance | Concentration |
|---|---|
| Calcium Chloride | 1100 mg/L |
| Papain FU | 22.00 mg/L |
| Dexamethasone 21-phosphate disodium | 44 μg/L or 10⁻⁷M |
| Scopolamine (Butyl bromide) | 4 mg/L |
| Gamma-Amino Butyric Acid | 0.06% w/v total |
| Butyric Acid | 0.06% w/v total |
| Retinoic Acid | 0.0020 mg/L |
| Hyaluronic Acid | 100.00 mg/L |
| Lauric acid (and/or Camphor oil) | 0.2% total in w/v |
| Ascorbic Acid | 400.00 mg/L |
| Carnithine | 0.05% w/v total |
| Physiological solution | q.s. per L of product |

### Composition Rationale

The present invention is based on the finding of a particular pro-proliferative stimulus exerted by the combination of calcium ions and proteolytic agents, preferably papain, optionally added with further active components, such as retinoic acid, scopolamine, dexamethasone, hyaluronic acid, butyric acid and/or GABA, lauric acid and/or camphor, ascorbic acid, carnithine or its salts and esters, on skin, mucosae, subcutis and connective tissues.

The activities of calcium ions and proteolytic enzymes, particularly papain, have already been widely illustrated in the present patent description.

Calcium ions are preferably introduced into the compositions of the invention in the form of calcium salts, preferably inorganic salts. Besides calcium chloride mentioned in the foregoing tables, we cite calcium gluconate, calcium phosphate, calcium nitrate and combinations thereof by way of example.

In addition to the already mentioned papain, collagenases (preferably type Ia, type II, type IV), serratiopeptidases, heparanases, elastases, bromelain, bradykinases, Clostridium peptidases, enzymes expressed by Lactobacillus acidophilus, enzymes expressed by the genus Aspergillus, proteases, aliinases, fibrinolysin, for example, may be used as proteolytic enzymes.

Polydeoxyribonucleotides (PDRN), resulting from the breaking down of nucleic acids, are very well tolerated molecules that enter the physiologic catabolism of nucleic acids. Polynucleotides are known to exert a physiologic stimulus towards cell proliferation and tissue repairing in damaged tissues. It is also known that the derivatives from the enzymatic break down of the polynucleotide chains (simple nucleotides, nucleosides, nitrogenous bases) are physiologically present in the extracellular environment and are useful trophic substrates for favouring cell regeneration and metabolic activity. In vivo, polynucleotides and nucleotides are used at tissue level both to improve cell activity and to protect and promote the physiological repairing and regenerating mechanisms.

Retinoic acid (vitamin A) and retinaldehyde (retinoid) enhance an appropriate tissue nutrition, leading to a gradual physiologic restoration of the microenvironment of cutis and subcutis (restoration of the correct pH and antioxidant capacity). Furthermore, more generally, the vitamins in the organism carry out several biological functions related to the complex cell differentiation process. Moreover, certain pathologies concerning cutis and subcutis (for example atrophies or cutaneous dystrophies) are caused by deficiency or unsuccessful absorption of vitamins.

Ascorbic acid (vitamin C) is added into some embodiments of the composition in that it promotes the regeneration (formation and maturation) of collagen in the damaged dermis and in the basal membrane.

Dexamethasone (a corticosteroid) and scopolamine (a parasympatholytic), used at doses that do not have any pharmacological effect, contribute to the normalisation of the loco-regional microenvironment, by promoting an ideal sodium-calcium ion exchange and favouring the adjustment to physiological values of the modified electrolyte concentration: they play an auxiliary role in the consolidation with regard to osmosis, viscosity and pH of the extracellular microenvironment in which cutis and subcutis live. Moreover, with the restoration of a physiological microenvironment it is possible to promote the activation of two repairing mechanisms in the organism: the attraction of stem cells from the circulatory stream and an accelerated specialisation of the stem cells residing in the skin towards a quick differentiation into mature cells committed to restore the damaged tissues. As an alternative to dexamethasone, any other glucocorticoid or corticosteroid may be used, such as betamethasone-disodium-phosphate, hydrocortisone, methylprednisolone, methylprednisolone sodium hemisuccinate, cortisone, cortisol, glycyrrhetic acid, natural or synthetic precursors or derivatives thereof.

As an alternative to scopolamine, other parasympatholytics may be used, such as adiphenine, aminocarbofluorene, anisotropine, anticholinesterases, atropine, benzatropine, cyclopentolate, clidinium, dicyclomine, dicycloverine, dioxyline, hexocyclium, ethaverine, glycopyrrolate, himbacine, ipratropium, mcn-a-343 (m-chlorophenyl-carbamoyl-oxybutinyl-trimethyl-ammonium-chloride), methyl-scopolamine, metocramine, mepenzolate, metanteline, muscarine, omatropine, oxyphencyclimine, oxyphenonium, oxotremorine, piperidolate, poldine, pipenzolate, pirenzepine, pirenzepine analogue (AF-DX 116), pralidoxine, propanteline, propanteline bromides, prifinium, thiemonium, thiotropium, tolterodine, tripitramine, tropicamine, trospium, scopolamine, or scopolamine butyl bromide, joscine N-methyl bromide, the derivatives and the natural and synthetic alkaloids of the above listed substances, as well as combinations thereof.

Butyric acid, also called n-butanoic acid, is a fatty acid, that is a monocarboxylic aliphatic acid and as such is a constitutive ingredient of almost all of the complex lipids and vegetable and animal fats. Butyric acid was credited with the ability of inhibiting the functions of the histone deacetylase enzyme. Therefore, it is able to favour an acetylated state of the histones in the cell. Acetylated histones have a lower affinity for DNA compared to non-acetylated histones, because of the neutralisation of the electrostatic charge interactions. It is generally believed that transcription factors can not gain access to regions wherein the histones are tightly associated with DNA (such as the heterochromatin, which is not acetylated). Thus, butyric acid is thought to enhance the transcriptional activity for those factors typically silenced or inhibited by the action of deacetylases. In the formulations, which form the subject of the present invention, when it is inserted at low concentrations it appears to bring about a gradual physiologic restoration of the microenvironment, especially of the subcutaneous one, actively counteracting the blemishes caused by cellulite and promoting collagen biosynthesis from loco-regional fibroblasts (anti-ageing effect).

Gamma-Amino butyric acid (GABA), or 4-aminobutanoic acid according to the IUPAC nomenclature, in vivo is released by neurons of the local circuits in the brain, which display a small neuronal body and branch at short distances, mostly forming axo-axonic synapses with the projecting (excitatory) neurons. GABA is a ubiquitous messenger and the activation or the antagonism at its receptors is the mechanism of action for a great number of sedative, myorelaxant, hypnotic, antiepileptic drugs, etc. In vitro, it assists the activity of butyric acid by promoting the fibroblast differentiation process and the depositing of collagen into the subcutaneous panniculus.

The effectiveness of lauric acid and camphor oil can be assumed to be induced by the combination of different mechanisms of action:
1) local selective antagonism on the binding between dihydrotestosterone and the androgen receptor;
2) inhibition of 5-alpha-reductase, an enzyme involved in the transformation of testosterone into dihydrotestosterone, which stimulates cell proliferation;
3) anti-inflammatory and anti-edemigenous action, as demonstrated by reduced capillary permeability induced by histamine;
4) anti-estrogenic effect, caused by a big decrease in estrogen receptors;
5) loco-regional antiseptic effect with mycobacteriostatic action.

The best known activities for carnithine and its salts and esters are the mitochondrial beta-oxidation of long-chain fatty acids (from the biochemical point of view, carnithine acts by taking part in a complex mechanism called carnithine acyl-CoA transferase) and the regulation of glucose usage. In skin ageing, increase in the size of adipocytes localised in the subcutaneous tissue and reduction in numbers and size of residential fibroblasts can be observed; carnithine and its salts and esters seem to be able to modulate such phenomena in a histological and biochemical way. It has been demonstrated that the fibrous component of the dermal connective tissue undergoes changes in the numbers and thickness of collagen fibres (increase in collagen cross-linking) and in the metabolic activity of polysaccharides in the dermis. Carnithine and its esters play a role in this case too by stabilising the membranes, which is essential for the cell repairing processes and for the functionality of the cell itself. Endogenous factors (the production of oxygen free radicals is one of the main causes of ageing) and environmental factors (solar radiation) add to the above-mentioned changes, the said factors causing both an acceleration of the skin ageing processes and a pathological transformation of the process itself. The optimal ability of the reaction of cells to noxious stimuli during ageing is attained via the maintenance of energy production and osmotic equilibrium. Carnithine is involved in the intermediate metabolism of lipids and carbohydrates, which is essential to the function of cells.

Hyaluronic acid (preferably in the form of sodium salt or lysinate), which is physiologically present approximately at 1% in the amorphous matrix of a connective tissue, is designed to restore the hydration, turgidity, plasticity and viscosity ratio of damaged skin. In vivo, hyaluronic acid is also able to act as a cementing substance and anti-collision molecule as well as an efficient lubricant, preventing cell and tissue damage by physical stresses. This molecular scaffold guarantees a strong support and the regeneration of the subcutaneous tissue. Furthermore, a filter is created against the free diffusion of particular substances, bacteria, infective agents in the tissue.

As an alternative to hyaluronic acid, other glycosaminoglycans may be used, such as for example chondroitin sulphates.

The cosmetic, pharmaceutical and medical device compositions according to the present invention can also comprise further accessory elements such as excipients and carriers, the selection and use thereof falls within the skills of the person of ordinary skill in the art without requiring the exercise of any inventive activity.

The culture media according to the invention too can comprise further ingredients, such as for example the usual inorganic salts, sugars, peptides, amino acids and vitamins required for the maintenance and/or growth in culture of mammalian cells, as well as the optional antibiotics and/or antimicrobial agents required to avoid culture contamination.

Among the amino acids that may be present in the compositions of the invention, methionine, cystine, N-acetylcysteine, cysteine, glycine, leucine, isoleucine, proline, glutamine, arginine, glutamic acid, histidine, histidine-HCl, lysine, lysine-HCl, phenylalanine, serine, threonine, tryptophan, tyrosine, tyrosine disodium salt, valine, proline, and hydroxyproline can be mentioned by way of example. Such amino acids are often used in mixtures comprising a high number of different amino acids. Besides the amino acids, the compositions of the invention may also comprise peptides and proteins, such as glutathione, collagen, elastin, wheat extract, and the like.

Among the vitamins that may be present in the compositions of the invention, we mention, by way of example, retinoic acid, retinaldehyde, retinol, alpha-tocopherol, beta-carotene, ascorbic acid, pantothenic acid, D-calcium pantothenate, pyridoxine, pyridoxine-HCl, folic acid, niacinamide, riboflavin, cobalamine, para-aminobenzoic acid and biotin.

Examples of cell culture solutions are for instance RPMI 1640 (cell culture medium), DMEM-LG (cell culture medium), AIM-V (cell culture medium), high glucose concentration modified D-MEM (cell culture medium), EBM (cell culture medium), human albumin, FBS (foetal bovine serum for cell cultures), F12 (cell culture solution containing a complete amino acid source), HANK's solution (cell culture solution containing sodium bicarbonate).

Finally, for the category of antibiotics and antimicrobials we mention, by way of example, gentamycin, penicillin, streptomycin, cyprofloxacin, levofloxacin, methronidazole, chlorhexidine, amphotericin B, fluconazole, itraconazole, triazole antimycotics, silver (it has a bacteriostatic activity).

### EXAMPLE 1

### Biopsies and Prototype Solutions

### Biopsies

All the biopsy samples under examination are listed as follows:
biopsies from human scalp;
biopsies from cutis and subcutis;
biopsies from parodontal tissue.

All the samples have been washed three times with physiological solution and antibiotics (100 units/ml penicillin+100 μg/ml streptomycin+160 mg/L gentamycin, 0.2 mg/ml fluconazole) for 10 min at room temperature.

The biopsies were then sectioned into three parts (two controls and one sample to be treated for each patient) and suspended in a Q-BASE Infus culture medium solution in 15 cm dishes (Lab-Tek chamber slides, Nunc, Kamstrup, Denmark).

Two types of controls were prepared, one negative control (1) exclusively treated with physiological solution and antibiotics (as described above), and one negative control (2) treated with common cell culture media.

1. The control biopsy samples were suspended in physiological solution in 15 cm dishes (Lab-Tek chamber slides, Nunc, Kamstrup, Denmark).

2. The control biopsy samples were then placed into 15 cm dishes (Lab-Tek chamber slides, Nunc, Kamstrup, Denmark) in D-MEM medium supplemented with:
10% FBS (Celbio, Milan, Italy)
100 units/ml penicillin
100 μg/ml streptomycin
160 mg/L gentamycin (Schering-Plough, Milan, Italy)
0.2 mg/ml fluconazole (Pfizer Italia S.r.l.),
2 mM L-glutamine (Life Technologies; growth medium).

All the samples were placed into a Heraeus thermostatically controlled incubator at a temperature of 37° C. in an atmosphere containing a constant flow of 8% CO₂ (v/v in air).

All the biopsy tissues used in culture constitute a possible optional co-conditioning support for the three-dimensional growth of the cell samples studied.

### Staining Protocol

After three washes for 10 min at room temperature in PBS (pH 7.4), the samples were resuspended in a 4% fixing paraformaldehyde solution in D-MEM (Gibco) at pH 7.4, for 1 hour at room temperature. All the biopsies under examination were treated with Alcian blue. This dye is made up by a group of water-soluble polyvalent basic colours. The blue colour is due to the presence of copper in the molecule. The Alcian blu in solution with PBS (pH 7.4) at 1% w/v final concentration is added to a solution of 3% acetic acid (pH 2.5). This composition, after a 2 hour incubation at room temperature, colours indelibly by binding the acidic mucopolysaccharides and both the sulphonated and carboxylated glycoproteins. Specific controls were set up for each sample. All the samples were washed 3 times with PBS (pH 7.4) at room temperature for five minutes and then viewed under a light microscope. A clear increase in type 2 collagen and type 4 collagen is detected, which turn blue, in the samples treated with the Q-BASE culture medium solution compared to control 1 and control 2.

### Results

### Staining by the Alcian Blue Colorimetric Method

Control 1 treated with physiological solution: very faint background staining barely detectable (score=+/−).
Control 2 treated with common D-MEM culture medium for biopsies, supplemented with 10% FBS, as described above. A very faint diffused background staining in light blue (Alcian blue) is detected (score=++).
Sample treated with the Q-BASE culture medium solution. It can be seen that the cells, in which a re-deposition of mucopolysaccharides and glycoproteins has been induced, get clearly coloured with Alcian blue, growing in layers one on top of the other (score=+++++).

### Western Blot

The samples were subjected to phenotypic analyses by Western blotting for the following markers: anti-type II collagen (Santa Cruz Biotechnology, America, California), anti-type IV collagen (Santa Cruz Biotechnology, America, California), anti-cytokeratin-1, -5, -10, -14 (Santa Cruz Biotechnology, America, California). After five washes, the filters were incubated with the relevant secondary antibodies (1:1000) conjugated to horseradish peroxidase (HRP, Santa Cruz Biotechnologies Inc., Santa Cruz, Calif. USA) for 1 hr at room temperature, as reported in the following tables 4 to 10.

Characterisation of Cutaneous Tissue Treated with Q-Base Culture Medium Versus Non-Treated Controls

The results pertaining to the expression of type II collagen, type IV collagen and cytokeratins 1, 5, 10 and 14 have been expressed as a quantitative scale, as follows:

TABLE 3

| Markers | Control 1 | Control 2 | Sample |
|---|---|---|---|
| type II collagen | −/+ | ++ | +++++ |
| type IV collagen | −/+ | ++ | +++ |
| cytokeratin 1 | ++ | ++++ | +++++ |
| cytokeratin 5 | +++ | ++++ | +++++ |
| cytokeratin 10 | ++ | ++++ | +++++ |
| cytokeratin 14 | +++ | ++++ | +++++ |

Legend
−−− = no band
−/+ = faint presence of a band
+ = presence of a thin band
++ = presence of a medium band
+++ = presence of a broad band
++++ = presence of a high band
+++++ = presence of a spread band

### EXAMPLE 2

### In Vivo Clinical Studies

### Q-BASE_CREAM Formulation.

### In Vivo Clinical Studies I.

A clinical study was performed, which was designed to assess the tolerability and therapeutic effectiveness of the product designated as Q-BASE-CREAM in the regeneration and repairing of cutis and subcutis.

The present study was carried out on a sample of eight dogs and two cats of different breeds and size that exhibited skin wounds due to different pathologies (canidae: lacerated and contused wounds, surgical wound dehiscence, scrotal ulcerous dermatitis, pyotraumatic dermatitis, acral lick dermatitis and burns; felines: face and neck self-traumatic dermatitis).

At the examination for enrolment, subjects suffering from the abovementioned wounds were selected. An affixing cytological examination was performed to assess the presence of existing over-infections.

In the case of detection of concurrent infections, a systemic antibiotic was prescribed.

The affected part was treated by topical application of physiological solution so as to mechanically remove the presence of exudate and crusts and then the Q-BASE-CREAM preparation was applied twice a day.

The animal was subjected weekly to a clinical check-up until it healed and underwent a cytological examination and an estimation of the extent and depth of the skin wound.

### Case 1

Female 9 year old Boxer operated on for grade II mastocytoma in the right axillary area. On day fifteen after the operation, after the removal of the suturing stitches (reabsorbable Vicryl 4/0), the dog showed a partial dehiscence of the wound due to self-traumatism.

Therapy. Marbofloxacin was administered per os at 2.5 mg/Kg for 7 days. The wound was cleansed with physiological solution and by application of the Q-BASE-CREAM preparation twice a day.

Check-ups. After one week, a granulation tissue was observed with a decrease of the wound up to about half of its original size, whereas cytologically, no extra- or intra-cytoplasmic bacterial colonies were present. At the end of the second week, a complete healing of the wound could be observed, with the formation of a stable scar tissue.

Clinical outcome. During the following check-up a month after the suspension of the treatment, the scar appeared whitish, with a normal strength and texture.

### Case 2

A male 5 year old Shi-t-zu was taken to a clinical examination because of a serious scrotal ulcerous dermatitis.

Therapy. Initially, prednisone was administered at 1 mg/kg for three days in order to reduce the exudation and marbofloxacin was administered per os at 2.5 mg/Kg for 7 days, cleansing the wound daily with povidone-iodine. After one week, an improvement of pain and edema and the disappearance of the infection could be observed, but there was still a difficulty in the healing of the ulcerative skin tissues, therefore the wound was cleansed with physiological solution and by application of the Q-BASE-CREAM preparation twice a day.

Check-ups. After one week, the ulcerative wounds were shown to be reduced to small superficial erosions (corresponding to 20% of the initial wound), cytologically, no bacteria were present; at the end of the second week, a complete healing of the scrotal skin could be observed with a progressive pigmentation of the skin.

Clinical outcome. During the following check-up two weeks after the suspension of the treatment, the skin appeared normalised with re-growth of the hair.

### Case 3

A male 5 year old Alsatian was taken to a clinical examination because of a self-traumatic and exudative wound on its back (lombosacral area). The case history is referable to a secondary pyotraumatic dermatitis from an allergic dermatitis due to a flea bite.

Therapy. Initially, the dog was administered with an adulticide spot-on anti-parasitic therapy (fipronil) and prednisone at 1 mg/kg for 2 days, in order to reduce the exudation and the itching, on the first day the wound was cleansed with povidone-iodine, then it was cleansed with physiological solution and the Q-BASE-CREAM preparation was applied thereon twice a day during the remaining 7 days.

Check-ups. After one week, an improvement of pain and edema, the disappearance of the infection and a complete healing of the tissue could be observed.

Clinical outcome. During the following check-up two weeks after the suspension of the treatment, the skin appeared normalised with re-growth of new hair.

### Case 4

A female 11 year old Alsatian was taken to a clinical examination because of a lesion of the peroneal nerve and, as a consequence, the leaning on the dorsal part of the paw, and self-traumatic wounds from skin licking. A surgical correction was performed by transposing the toes' flexor tendon that was anchored to the toes' extensor tendon, improving the posture and enabling the distal end of the limb to lean on correctly. Despite the motor recovery, the skin wounds from chronic licking remained. The case history is referable to a chronic self-traumatic dermatitis complicated by secondary superficial infections.

Therapy. The dog was administered with a systemic antibiotic therapy with enrofloxacin at 5 mg/kg per os for three weeks. On the first day, the wound was cleansed with povidone-iodine, then it was cleansed with physiological solution and the Q cream preparation was applied thereon twice a day for 14 days.

Check-ups. After one week, an improvement of the infection and a 50% healing of the tissue could be observed. After two weeks, the disappearance of the infection and a further decrease of the wounds could be observed.

Clinical outcome. During the following check-up two weeks after the suspension of the treatment, a complete healing of the skin could be observed, with a decrease in the dermal fibrosis.

### Case 5

A male 6 year old mongrel was subjected to examination due to a road trauma with multiple skin wounds of the pelvis and hind limbs.

Therapy. The dog was administered with cefalexin per os at 25 mg/kg for 7 days. The wound was cleansed with physiological solution and the Q cream preparation was applied thereon twice a day.

Check-ups. After 24 and 36 hours, formation of crusts and the presence of superficial sepsis on the medial side, and wound suppuration in the iliac area were observed. After 50 hours, the formation of a granulation tissue was detected, with excellent wound reduction (less than 50%). After 72 hours, healing was observed on the medial side (less than 10% of wounds). After 90 hours, a complete healing of the deep layer occurred. After one week, a complete healing of the wounds could be observed, with the formation of a stable scar tissue.

Clinical outcome. During the following check-up a month after the suspension of the treatment, the scars appeared whitish, with a normal strength and texture.

### Case 6

Male 11 year old Yorkshire terrier, operated on for hepatoid gland epithelioma in the left perianal area. By the eighth day from the operation, the dog showed a partial dehiscence of the surgical wound due to self-traumatism.

Therapy. Marbofloxacin was administered per os at 2.5 mg/Kg for 7 days. The wound was cleansed with povidone iodide-iodine and by application of the Q-BASE-CREAM preparation twice a day.

Check-ups. After one week, a granulation tissue was detected, with wound reduction up to about 10% of its original size, and at the end of the second week, a complete healing of the wound could be observed, with the formation of a stable scar tissue.

Clinical outcome. During the following check-up a month after the suspension of the treatment, the scar appeared whitish, with a normal strength and texture.

### Case 7

A female, 12 year old, diabetic Yorkshire terrier was taken to a clinical examination because of the presence of multiple burns on its back caused by a burning (accidental wound).

Therapy. Marbofloxacin was administered per os at 2.5 mg/Kg for 21 days. Its hair was shaved off and the wound was cleansed with povidone iodide-iodine, followed by application of the Q-BASE-CREAM preparation twice a day.

Check-ups. After one week, a granulation tissue was detected, with wound reduction up to about 20% of its original size, and at the end of the second week, a complete healing of the wound could be observed, with the formation of a stable scar tissue.

Clinical outcome. During the following check-up a month after the suspension of the treatment, the scar appeared whitish, with a normal strength and texture.

### Case 8

A female, 10 year old, mongrel terrier was taken to a clinical examination because of the presence of skin wounds on its flanks and back, which appeared approximately one week after a sterilisation surgical operation, secondary to burns from a thermo-electric effect (from thermocautery).

Therapy. Enrofloxacin was administered per os at 5 mg/Kg for 21 days. Its hair was shaved off, the crusts were removed and the wound was cleansed with povidone iodide-iodine, followed by application of the Q-BASE-CREAM preparation twice a day.

Check-ups. After one week, a granulation tissue was observed with a decrease of the wound up to about half of its original size and a week later, a complete healing was detected.

Clinical outcome. During the following check-up a month after the suspension of the treatment, a post-cicatricial alopecia was observed.

### Case 9

A male 2 year old European cat was taken to a clinical examination because of a self-traumatic and exudative wound on its back in the interscapular area. The case history is referable to a secondary pyotraumatic dermatitis from vaccine inoculation.

Therapy. The cat was administered with a systemic antibiotic therapy with amoxicillin and clavulanic acid (12.5 mg/kg bid for 7 days). On the first day, the wound was cleansed with povidone-iodine, then it was cleansed with physiological solution and the Q-BASE-CREAM preparation was applied thereon once a day during the remaining 7 days, keeping the animal bandaged in order to prevent further self-wounding.

Check-ups. After one week, a complete healing of the tissue was detected.

Clinical outcome. During the following check-up two weeks after the suspension of the treatment, the skin appeared normalised with re-growth of new hair.

### Q-BASE_CREAM Formulation

### In Vivo Clinical Studies II

A clinical study was performed, which was designed to assess the therapeutic effectiveness of the product designated as Q-BASE-CREAM in the regeneration and repairing of cutis and subcutis.

During the period comprised from March to May 2007, a mixed group of volunteers consisting of human (two women and one man) and veterinary (two dogs) patients were included in the clinical study.

### Case 1

57 Year old African man.

### Medical History and Therapeutic Protocol

The patient had a traumatic scratch wound on his face occurred 48 hours before, treated solely with 10% povidone-iodine. At the time of the inclusion (day 0), the Q-BASE-CREAM was applied twice a day for 21 days.

Day 0. Presence of erosions, ulcerations and perilesional depigmentation, associated with a serocellular crust with an approximately 2 cm diameter irregular area.

Day 7. The serocellular crust present at the time of the examination had decreased and the depth of the lesion was reduced up to 50%.

Day 14. Small superficial, linear, depigmented scars remained.

Day 21. During the final examination, a flattening of the scars and a progressive skin re-pigmentation were detected.

### Summary of the Clinical Evolution

The patient exhibited predisposition to the formation of excess scars. The application of the Q-BASE-CREAM overcame this problem.

### Case 2

41 Year old Caucasian woman.

### Medical History and Therapeutic Protocol

The patient showed a surgical suture dehiscence (operation on a C6-C7 cervical hernia) and a contact allergic reaction (patch). She was administered with an injectable systemic therapy consisting of a single dosage of cephalosporin.

Day 1. Presence of erythema and contact dermatitis. The surgical wound, approximately 6 cm in length, exhibited dehiscence of the central area, accompanied by the presence of purulent material.

Day 7. Following local disinfection with povidone-iodine alone, no changes in the lesion occurred.

Only a progressive improvement in the contact dermatitis was detected.

Day 14. Silver sulfadiazine was then locally applied as an ointment once a day for 7 days prior to the clinical examination.

No clinical improvement whatsoever was observed at this time.

Day 21. Upon examination, because there was no further healing of the wound, the Q-BASE-CREAM was applied every 12 hours.

Day 30. Upon examination, the wound showed healing and scar tissue.

### Summary of the Clinical Evolution

First week. Dehiscence of the suture and retraction of the wound edges; on the second week, the presence of exudative purulent material and a persistence of lack of healing were noted.

The replacement of the topical therapy by the Q-BASE-CREAM allowed for the wound closure at the 4^{th} week after applying it for 9 days.

### Case 3

17 Year old Caucasian woman.

### Medical History and Therapeutic Protocol

The patient was taken to a clinical examination because of the presence of a 1 cm keloid in the pre-tibial area that she'd had for 4 years. She was treated with a topical therapy by daily applications of the Q-BASE-CREAM for 2 months.

Day 0. The wound in the tibial area was approximately 1 cm in size, hyperplastic, and showed a modest perilesional erythema.

Day 60. Upon examination, the wound was decreased by 60% since the beginning of the therapy.

### Summary of the Clinical Evolution

In this case, a progressive decrease in the keloid thickness occurred (60%).

### Case 4

Report. Male 5-month-old Golden retriever.

Medical history. Problems of spontaneous skin lacerations accompanied by secondary infections and healing disorders since birth. Prior to the consultation, the dog had been subjected to several attempts at surgical revision and suturing of the wounds. The patient had previously undergone many therapies: tetracyclines per os for 1 month associated with a cortisone depot, local disinfections in the lesion areas with aminosidine sulphate+prednisolone and clostebol acetate. Despite the treatments, no significant results had been observed. Clinically, the dog exhibited cutaneous hyperextensability. Multiple skin biopsies were performed, which revealed an alteration in the collagen fibres consistent with Ehlers-Danlos Syndrome.

Day 0. The skin wounds and lacerations were treated with polyvynilpyrrolidone twice a day and Q-BASE-CREAM occlusive bandaging of the limbs and Q-BASE-CREAM free application, without bandages, into the ischial area.

A systemic antibiotic therapy with 12.5 mg/kg bid amoxicillin and clavulanic acid was simultaneously given in order to control the secondary infections.

Day 30. After 30 days, a re-epitheliation of all the wounds treated with Q-BASE-CREAM was observed. Small erosive and ulcerous areas remained in zones where the patient leans on (elbows).

The use of the Q-BASE-CREAM allowed to accomplish healing of extensive skin wounds in only 30 days, the which wounds had been complicated by over-infections since months as a result of the collagen genetic disease present since birth.

### Case 5

Report. Male 10-year-old WHWT dog.

Medical history. Since one year the dog was exhibiting interdigital hamartomas (nodular skin areas presenting fibro-annexial dysplasia) in the forelegs, which had progressively increased in diameter in the last 2 months, until reaching the size of approximately 4 cm in diameter. The dog was subjected to surgical removal and antibiotic therapy with 20 mg/kg/bid cefalexin for 15 days. 4 Days later, during the check-up, an ischemic-based skin necrosis area (2.5 cm diameter) was detected, caused by a too tight bandage, as well as a partial dehiscence of the surgical suture in the right foreleg.

Day 0. The dog was subjected to disinfection with povidone-iodine and application of the Q-BASE-CREAM into the interdigital area once a day, with an occlusive bandage until examination.

The systemic antibiotic therapy with cefalexin was continued at the same dose.

Day 15. During the check-up, a remission of the wound was noted.

The application of the Q-BASE-CREAM allowed to attain a re-epitheliation of the necrotic skin area in a short time period, with a normal healing of the surgical wound.

### General Conclusions

In every patient, a quicker tissue healing was observed compared to other previously used topical therapies.

### QParodontal-BASE and QParodontal-BASE-Gel Formulation

### In Vivo Clinical Studies III.

A clinical study was performed, which was designed to assess the tolerability and therapeutic effectiveness of the product designated as QParodontal-BASE and QParodontal-BASE-Gel in the regeneration of the oral mucosae and parodontal tissue. The present study was carried out on a sample of 5 veterinary cases.

### Case 1

3-Year-old white miniature poodle.

Dental care session prior to a canine exposure.

Clinical condition: presence of tartar mostly on the upper dental arch, particularly on the premolars and molars; moderate gingivitis; no difficulty in chewing.

Therapy: detartrasis and polishing; therapy starting with the gel twice a week for thirty days.

Results: excellent outcome of the therapy, with improvement of the gingivitis and disappearance of the halitosis.

### Case 2

8-Month-old male miniature poodle.

Clinical condition: mild gingivitis caused by the recent loss of the deciduous teeth and by a mainly moist diet; moderate halitosis.

Therapy: no detartrasis treatment; therapy starting with the gel once a week for thirty days.

Outcome: moderate improvement of the gingivitis; excellent improvement of the halitosis.

### Case 3

9-Year-old male castrated tabby cat.

Clinical condition: serious halitosis; difficulty in chewing; parodontopathy with consequent loosening of the upper and lower premolar and molar teeth.

Therapy: extraction of the loose teeth; detartrasis; polishing; therapy starting with the gel four times a week for thirty days.

Outcome: clear improvement of the gingivitis; disappearance of the halitosis; disappearance of the chewing difficulties.

### Case 4

6-Year-old female British shorthair cat. From a farm, awaiting adoption, having ended its reproduction career.

Clinical condition: presence of serious gingivo-stomatitis and fasciitis; total impossibility of chewing due to intense pain; presence of bleeding and round-shaped gingival outgrowths around the lower molars and premolars.

Diagnostic tests: normal biochemical profile. FiV and FelV tests proved negative.

Cytological examination: lymphoplasmacytic stomatitis.

Histological examination: fibromatous epulis with plasma cell inflammation.

Treatment: extraction of the lower molars around the gingival outgrowths; therapy with the gel four times a week for thirty days.

Outcome: cessation of the pain and re-establishment of a correct chewing; improvement of the halitosis and slight improvement of the inflammation; moderate regression of the gingival outgrowths.

### Case 5

11-Year-old male mongrel.

Clinical condition: extremely serious halitosis; difficulty in chewing; serious parodontopathy with consequent loosening of the upper and lower premolar and molar teeth.

Therapy: extraction of the loose teeth; detartrasis; polishing; therapy starting with the gel four times a week for thirty days.

Outcome: clear improvement of the gingivitis; disappearance of the halitosis; disappearance of the chewing difficulties.

### QNasal-BASE Formulation.

### In Vivo Clinical Studies Iv.

A clinical study was performed, which was designed to assess the tolerability and therapeutic effectiveness of the product designated as QNasal-BASE in the regeneration of the nasal mucosae. The present study was carried out on a sample of 2 clinical cases.

Two patients were treated, wherein the said patients were affected by relapsing nasal polyposis, which had already been operated on.

### Case 1

54-Year-old

Caucasian woman.

The patient had been operated on three times in the last 10 years because of a relapsing ethmoidal polyposis.

The patient reported no allergies to all of the antigens tested (no positivity to the epicutaneous test); she was not taking acetylsalicylic acid or FANS. During the year 2006 and the first months of 2007, she suffered from relapsing nasal polyposis (of the ethmoidal cells), non-responding to any common pharmacological therapy.

The patient was treated for thirty days; she showed a clear decrease in the symptoms, with her great satisfaction. The size of the polyps appeared decreased by 70% at the endoscopic examination. The patient continues the treatment.

### Case 2

60-Year-old Caucasian man.

Operated on three years ago because of a relapsing nasal polyposis. No allergies. He was not taking drugs. He was proposed another surgical operation because of a violent symptomatology.

Instead, he begins treatment with the QNasal-BASE with excellent subjective and objective results, the size of the polyps appeared decreased by 50% after 20 days of treatment.

### REFERENCES

1. Robinson M, Reynolds A J, Gharzi A, Jahoda C A. In vivo induction of hair growth by dermal cells isolated from hair follicles after extended organ culture. J Invest Dermatol. 2001 September; 117(3):596-604)

1-21. (canceled)

22. A composition comprising, in a physiologically acceptable vehicle,
amino acids, sugars, salts and vitamins for an in vitro culture of eukaryotic cells or tissues,
at least one proteolytic enzyme, and
a calcium salt,
wherein the calcium salt is comprised in an amount suitable to provide a concentration of calcium ions of at least 3.6 mM, and
wherein the composition is capable of inducing a regenerating activity on cells and tissues, the regenerating activity accelerating tissue repairing and allowing maintenance of cultured tissue biopsies under viable conditions for at least 6 months.

23. The composition according to claim 22, wherein the proteolytic enzyme is selected from the group consisting of papain, collagenase, Serratiopeptidase, heparanase, elastase, bromelain, bradykinase, Clostridium peptidase, proteolytic enzymes expressed by Lactobacillus acidophilus, proteolytic enzymes expressed by the genus Aspergillus, protease, aliinase and fibrinolysin.

24. The composition according to claim 23, wherein the proteolytic enzyme is papain.

25. The composition according to claim 23, wherein the proteolytic enzyme is comprised in a concentration within a range of 0.1 μg/L to 50 mg/L.

26. The composition according to claim 25, wherein the proteolytic enzyme is comprised in a concentration within a range of 2 mg/L to 30 mg/L.

27. The composition according to claim 22, wherein the concentration of calcium ions is in a range of 7 to 27 mM.

28. The composition according to claim 27, wherein the calcium salt is selected from the group consisting of calcium chloride, calcium gluconate, calcium phosphate, and calcium nitrate.

29. The composition according to claim 27, wherein the calcium salt is calcium chloride.

30. The composition according to claim 22, further comprising one or more components selected from the group consisting of polydeoxyribonucleotide (PDRN), mucopolysaccharides, parasympatholytics, corticosteroids, glycyrrhetic or glycyrrhetinic acid and its salts, retinoic acid, retinoids, retinaldehyde, silver proteinate, and combinations thereof.

31. The composition according to claim 30, wherein the composition comprises a corticosteroid and the corticosteroid is dexamethasone.

32. The composition according to claim 30, wherein the composition comprises a parasympatholytic and the parasympatholytic is selected from the group consisting of adiphenine, aminocarbofluorene, anisotropine, anticholinesterases, atropine, benzatropine, cyclopentolate, clidinium, dicyclomine, dicycloverine, dioxyline, hexocyclium, ethaverine, glycopyrrolate, himbacine, ipratropium, mcn-a-343 (m-chlorophenyl-carbamoyl-oxybutinyl-trimethyl-ammonium-chloride), methyl-scopolamine, metocramine, mepenzolate, metanteline, muscarine, omatropine, oxyphencyclimine, oxyphenonium, oxotremorine, piperidolate, poldine, pipenzolate, pirenzepine, pirenzepine analogue (AF-DX 116), pralidoxine, propanteline, propanteline bromides, prifinium, thiemonium, thiotropium, tolterodine, tripitramine, tropicamine, trospium, scopolamine, or scopolamine butyl bromide, joscine N-methyl bromide; derivatives and natural and synthetic alkaloids thereof.

33. The composition according to claim 30, wherein the composition comprises a parasympatholytic and the parasympatholytic is scopolamine.

34. The composition according to claim 22, wherein the composition is an in vitro culture medium for cells or a tissue.

35. The composition according to claim 34, wherein composition is a medium designed to maintain tissue biopsies under viable conditions.

36. The composition according to claim 34, wherein the tissue is a tissue biopsy, and the tissue biopsy is a biopsy from cutaneous, subcutaneous, mucosal, lymphatic or scalp tissues.

37. The composition according to claim 22, wherein the composition is a regenerating and/or healing pharmaceutical composition or medical device in a freeze-dried, cream, gel, foam, powder, patch, film, coating or injectable form.

38. The composition according to claim 22, wherein the composition is an anti-aging, anti-wrinkle, anti-blemish, moisturizing, nourishing, elasticising, soothing, regenerating, anti-cellulite and/or anti-stretch mark cosmetic composition in a cream, gel, serum, emulsion, patch, spray, powder, foam or make up form.

39. A method of culturing a tissue biopsy, the method comprising
maintaining the tissue biopsy under viable conditions in an in vitro culture medium comprising the composition according to claim 22.

40. The method according to claim 39, wherein the tissue biopsy is a biopsy from a cutaneous, subcutaneous, mucosal, lymphatic or scalp tissue.

41. The method according to claim 40, wherein the tissue biopsy is maintained in continuous culture under viable conditions for at least 6 months.

42. The method according to claim 41, wherein the tissue biopsy is maintained in continuous culture under viable conditions for at least 3 years.

43. A method of healing and/or regenerating an animal tissue, the method comprising
applying to the animal tissue the composition of claim 22.

44. The method of claim 43, wherein the animal tissue is selected from the group consisting of cutaneous, subcutaneous and mucosal tissues.

45. A method for cosmetic treatment of skin and/or integumentary systems, the method comprising
applying the composition of claim 22 to the skin and/or integumentary system of a subject in need thereof.

46. The method of claim 45, wherein the applying results in an anti-ageing and/or regenerative treatment of the skin and/or integumentary system.
